(19) United States
(12) Patent Application Publication
(10) Pub. No.: US 2004/0265380 A1
(43) Pub. Date: Dec. 30, 2004

(54) Orodispersible effervescent tablets

(76) Inventors: Pascal Delmas, Outremont (CA); Jean-Marc Zuccarelli, Antibes (FR); Claire Mallard, Mougins Le Haut (FR); James W. McGinity, Austin, TX (US)

Correspondence Address: CAESAR, RIVISE, BERNSTEIN,, COHEN & POKOTILOW, LTD., 11TH FLOOR, SEVEN PENN CENTER, PHILADELPHIA, PA, 19103-2212, US

(21) Appl. No.: 10/475,208

(22) PCT Filed: Apr. 17, 2002
(86) PCT No.: PCT/FR02/01321

(30) Foreign Application Priority Data
Apr. 20, 2001 (FR) 01 05389

(51) Int. Cl. A61K 9/46

(52) U.S. Cl. 424/466

(57) ABSTRACT

The invention relates to an orodispersible effervescent tablet comprising at least one active ingredient, a mixture of excipients comprising at least one disintegrating agent, a diluting soluble agent, a luficating agent and, optionally, an internal dehydrating agent, a swelling agent, a permeabilizing agent, sweetening agents, flavouring agents and colorants, and effervescent granules based on a mixture which is essentially composed of an acidic agent, a heat extrudable binding agent and an alkaline agent and, optionally, a dehydrating agent and a lubricant, said granules being prepared by means of heat extrusion in the absence of of water and solvents, and said tablet disintegrating in the pharynx when it comes into contact with saliva in at least 60 seconds and preferentially in less than 40 seconds.

The present invention relates to orodispersible effervescent tablets.

For the purposes of the present invention an orodispersible tablet is a tablet capable of undergoing disaggregation in the mouth, in the absence of any chewing action, in less than 60 seconds, preferably in less than 40 seconds, in contact with the saliva, forming a suspension which is easy to swallow.

Certain patients, particularly the elderly and children, experience difficulties of deglutition such that it is difficult and, consequently, unpleasant for them to ingest tablets or capsules, even together with an intake of liquid. These difficulties result in the prescribed medicinal product not being taken and hence in the efficacy of the treatment being severely affected (H. Seager, 1998, J. Pharm. Pharmacol. 50, 375-382).

It is estimated that 50% of the population experiences such difficulties in swallowing tablets or capsules.

Pharmaceutical laboratories have therefore sought new pharmaceutical forms which are more pleasant to take.

Multiparticulate tablets with rapid disintegration have already been described by the Applicant Company in patents FR 99 02516, FR 97 09233, FR 98 14034, FR 92 08642 and FR 91 09245. These tablets comprise active substances in the form of coated microcrystals or coated microgranules, the coating being able to give them properties of taste masking, of stability enhancement of gastric fluid resistance or of modified release of the active principle.

However, in view of the ever-increasing demand for such orodispersible tablets, it is important to be able to provide new forms which can be adapted to any type of active principle and which are very easy and very pleasant to take.

Research by the Applicant Company allowed it to find that these features were obtainable when effervescent granules obtained by hot extrusion were incorporated into an orodispersible tablet comprising at least one active principle and a mixture of excipients comprising at least one disintegrant, a soluble agent and a lubricant and optionally a swelling agent, a permeabilizing agent, sweeteners, and flavorings.

The work of the Applicant Company described in patent U.S. Pat. No. 6,071,539 showed, in effect, that the effervescent granules on the one hand promote the disaggregation of the tablet and on the other hand give it advantageous organoleptic qualities, particularly from the standpoint of masking the taste of the active principle, when necessary, and of obtaining tickling sensations in the buccal cavity, which are particularly appreciated by young children.

The invention accordingly provides orodispersible effervescent tablets comprising:

at least one active principle,

a mixture of excipients comprising at least one disintegrant, a diluent soluble agent, a lubricant and optionally an internal dehydrating agent, a swelling agent, a permeabilizing agent, sweeteners, and flavorings and colorants,

and effervescent granules based on a mixture constituted essentially by an acidic agent, an alkaline agent and a heat-extrudable binder, and optionally a lubricant and an internal dehydrating agent,

said effervescent granules being prepared by hot extrusion in the absence of water and solvents,

and said tablets undergoing disaggregation in the buccal cavity in contact with saliva in less than 60 seconds and preferably in less than 40 seconds.

In the tablets in accordance with the invention the active principle is present either in the effervescent granules or outside the effervescent granules, in a mixture with the various excipients of the tablet.

According to one first embodiment in accordance with which the active principle is present in the effervescent granules, the tablet in accordance with the invention comprises:

a mixture of excipients comprising at least one disintegrant, a diluent soluble agent, a lubricant and optionally an internal dehydrating agent, a swelling agent, a permeabilizing agent, sweeteners, flavorings and colorants,

and effervescent granules based on at least one active principle and a mixture constituted by an acidic agent, an alkaline agent and a heat-extrudable binder and optionally a lubricant and an internal dehydrating agent,

said granules being prepared by hot extrusion in the absence of water and solvents.

According to a second embodiment, in accordance with which the active principle is not present in the effervescent granules, the tablet in accordance with the invention comprises:

a mixture of excipients comprising at least one disintegrant, a diluent soluble agent, a lubricant and optionally an internal dehydrating agent, a swelling agent, a permeabilizing agent, sweeteners and flavorings,

effervescent granules based on a mixture constituted by an acidic agent, an alkaline agent and a heat-extrudable binder and optionally a a lubricant and an internal dehydrating agent,

and at least one active principle,

said granules being prepared by hot extrusion in the absence of water and solvents according to the process described in patent U.S. Pat. No. 6,071,539.

According to another advantageous embodiment the active principle, whether incorporated or not in the effervescent granules, comprises a taste masking coating.

According to another advantageous embodiment, when the active principle is not incorporated in the effervescent granule, it comprises a coating.

This embodiment is particularly appropriate in cases where the active principle employed has a very unpleasant taste.

The coating which is employed in that case is a conventional taste masking coating which may consist of a polymer or of a mixture of polymers. This coating may further exhibit properties of stabilization, gastric fluid resistance, and modified release of the active principle.

The active principles employed in the tablets in accordance with the invention are therapeutic compounds, vitamins, mineral salts or nutritional supplements. The fields of application of the present invention may therefore be human and veterinary pharmacy and the neutracetic sector.

Accordingly, it is possible to use the following therapeutic compounds: synthetic antibacterial agents of pyridone-carboxylic acid type of low water-solubility, such as benzofloxacin, nalidixic acid, enoxacin, ofloxacin, amifloxacin, flumequin, tosfloxacin, piromidic acid, pipemidic acid, miloxacin, oxolinic acid, cinoxacin, norfloxacin, ciprofloxacin, perfloxacin, lomefloxacin, enrofloxacin, danofloxacin, binfloxacin, sarafloxacin, ibafloxacin, difloxacin, and their salts. Other therapeutic compounds which can be formulated in accordance with the invention include penicillin, tetracyclin, erythromycin, cephalosporins, and other antibiotics.

Other active principles which can be formulated in the form of tablets in accordance with the invention include antibacterial substances, antihistamines and decongestants, antiinflammatories, antiparasitics, antivirals, anxiolytics, morphine derivatives, serotonin antagonists, the levogyratory isomers of thyroxine, agents which lower the cholesterol level, alpha-adrenergic blockers, local anesthetics, antifungals, amebicides, or trichomonocides, analgesics, antiarthritics, antiasthmatics, anti-coagulants, anticonvulsants, antidepressants, anti-diabetics, antineoplastics, antipsychotics, antihyper-tensives, phenanthrene derivatives, antidiarrheals, diuretics, and muscle relaxants.

As antibacterial substances mention may be made of antibiotics of beta-lactam type, tetracyclines, chloramphenicol, neomycin, gramicidin, bacitracin, sulfonamides, antibiotics of aminoglycoside type, tobramycin, nitrofurazone, nalidixic acid and the analogues thereof and the antimicrobial combination fludalanine/pentizidone. As a phenanthrene derivative mention may be made of morphine sulfate. As an antidiarrheal mention may be made of loperamide hydrochloride. As an anxiolytic mention may be made of buspirone. As a diuretic mention may be made of hydrochlorthiazide. As a morphine derivative mention may be made of hydromorphone hydrochloride. As antihistaminics and decongestants mention may be made of perilamine, chlorpheniramine, tetrahydrozoline, and antazoline. As antiinflammatory medicaments mention may be made of cortisone, hydrocortisone, betamethasone, dexamethasone, fluocortolone, prednisolone, triamcinolone, indomethacin, sulindac and its salts, and the corresponding sulfide. As an antiparasitic compound mention may be made of ivermectin. As antiviral compounds mention may be made of acyclovir and interferon. As analgesic medicaments mention may be made of diflunisal, aspirin or acetaminophen. As antiarthritics mention may be made of phenylbutazone, indomethacin, silindac, its salts and corresponding sulfide, dexamethasone, ibuprofen, allopurinol, oxyphenbutazone or probenecid. As medicaments against asthma mention may be made of theophylline, ephedrine, beclomethasone dipropionate, and epinephrine. As anticoagulants mention may be made of heparin, bishydroxycoumarin, and warfarin. As anticonvulsants mention may be made of diphenylhydantoin and diazepam. As antidepressants mention may be made of amitriptyline, chlordiazepoxide, perphenazine, fluoxetin hydrochloride, protriptyline, imipramine, and doxepin. As an agent which lowers the cholesterol level mention may be made of simvastatin. As an alpha-adrenergic blocker mention may be made of ergotamine tartrate. As a serotonin antagonist mention may be made of sumatropin succinate. As a levogyratory isomer of thyroxine mention may be made of sodic levothyroxine. As antidiabetics mention may be made of insulin, somatostatin and its analogues, tolbutamide, tolazamide, acetchexamide, and chlorpropamide. As antineoplastics mention may be made of adriamycin, fluorouracil, methotrexate, and asparaginase. As antipsychotics mention may made of prochlorperazine, lithium carbonate, lithium citrate, thioridazine, molindone, fluphenazine, trifluoperazine, perphenazine, amitriptyline, and trifluopromazine. As antihyper-tensives mention may be made of spironolactone, methyldopa, hydralazine, clonidine, chlorothiazide, deserpidine, timolol, propranolol, metoprolol, prazosin hydrochloride, and reserpin. As muscle relaxants mention may be made of succinylcholine chloride, danbrolene, cyclobenzaprine, methocarbamol, and diazepam.

The pharmaceutical compounds present in the tablets in accordance with the invention may be present in the form of their pharmaceutically acceptable salts or any polymorphic form (racemic mixture, enantiomer, etc). By “pharmaceutically acceptable salts” are meant the derivatives of the compounds described in which the pharmaceutically active parent compound is converted into its basic or acidic salt; examples of pharmaceutically active salts include in particular the salts of organic or mineral acids with basic residues such as amines; alkali derivatives or organic salts of acidic residues such as carboxylic acids, and the like. The pharmaceutically acceptable salts include conventional nontoxic salts or quaternary ammonium salts of the parent compound, formed for example from nontoxic inorganic or organic acids. By way of example, such conventional nontoxic salts include those obtained from inorganic acids such as hydrochloric, hydrobromic, sulfuric, sulfonic, sulfamic, phosphoric, and nitric acid and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethanedisulfonic, oxalic, and isothionic acid, and the like.

The pharmaceutically acceptable salts of the present invention may be synthesized from the parent therapeutic compound which contains an acidic or basic fraction, by conventional methods. Generally speaking these salts can be prepared by reacting the free acidic or basic forms with a predetermined amount of the appropriate base or acid in water or in organic solvent or in a mixture of water and organic solvent. Preference is generally given to nonaqueous media. Lists of appropriate salts may be found in Remington's Pharmaceutical Sciences, 17th ed., Mack Publishing Company, Easton, Pa. 1985, p. 1418.

The expression “pharmaceutically acceptable” is employed here to refer to those compounds, materials, compositions and/or galenic forms which are, in accordance with medical judgment, appropriate for use in contact with human or animal tissue without excessive toxicity, irritation, allergic response or other problem or complication, in relation to a reasonable benefit/risk ratio.

The active principle employed in the tablets in accordance with the invention may also be a vitamin. As used herein, the term vitamin refers to any trace organic substance which is useful in the diet. The term vitamin includes in particular thiamine, riboflavin, nicotinic acid, pentotenic acid, pyridoxine, biotin, folic acid, vitamin B12, lipoic acid, ascorbic acid, vitamin A, vitamin D, vitamin E, and vitamin K. Likewise embraced by the term vitamin are their coenzymes. The coenzymes are specific chemical forms of the vitamins. The coenzymes include thiamine pyrophosphates (TPP), flavine mononucleotide (FMM), flavine adenine dinucleotide (FAD), nicotinamide adenine dinucleotide (AND), nicotinamide adenine dinucleotide phosphate (NADP), coenzyme A (CoA), pyridoxal phosphate, biocytin, tetrahydrofolic acid, coenzyme B12, lipoyllysine, 11-cis-retinal, and 1,25-dihydroxycholecalciferol. The term vitamin likewise embraces choline, carnitine, and alpha-, beta-, and gamma-carotenes.

As used herein, the term “mineral” refers to inorganic substances, metals, and the like which are required in the human diet. Accordingly the term “mineral” includes in particular calcium, iron, zinc, selenium, copper, iodine, magnesium, phosphorus, chromium and the like, and mixtures thereof.

The term “nutritional supplement” as used herein signifies a substance which has an appreciable nutritional effect when administered in small amounts. Nutritional supplements include in particular substances such as bee pollen, bran, wheatgerm, kelp, cod liver oil, ginseng, and fish oils, amino acids, proteins and mixtures thereof. Nutritional supplements may incorporate vitamins and mineral salts.

The amounts of active principle incorporated in each tablet may be selected according to the principles which are known in pharmacy. Generally an effective amount of active principle is incorporated.

The alkaline agent and the acidic agent are selected from those which are pharmaceutically acceptable such that in the presence of water they allow the release of a gas. The objective of the effervescent mixture here is a multiple one:

release of a pharmaceutically acceptable gas facilitating the rapid disaggregation of the tablet in contact with saliva and imparting desired organoleptic properties (taste masking, tickling sensation, etc.)

inducement of a buccal micro-pH which promotes the desired organoleptic properties by influencing, for example, the solubility of the active principle so as to lessen its perception in the buccal cavity or by inducing a pleasant slightly acid taste.

The volume of gas given off is comprised between 2 and 60 cm³ and preferably between 5 and 40 cm³.

Above 60 cm³ the volume induced is too great and becomes unpleasant within the buccal cavity. This feature differentiates the present invention from conventional effervescent tablets intended for dissolving in a glass of water, for which the volume of gas is generally greater, from 100 to 300 cm³ depending on the format and on the composition of the tablet.

The acidic agent is a proton donor compound which is able to react with an alkaline agent in order to form a gas which causes the liquid in which said gas is released to effervesce.

The acidic agent may be constituted by any mineral or organic acid, in free acid, acid anhydride or acid salt form.

According to one advantageous embodiment the acid in solid form at room temperature has a pH of less than or equal to 4.5.

This acid is selected from the group comprising, in particular, tartaric acid, citric acid, maleic acid, fumaric acid, malic acid, adipic acid, succinic acid, lactic acid, glycolic acid, alpha hydroxy acids, ascorbic acid and amino acids, and also the salts and derivatives of these acids.

The alkaline agent is constituted by a compound capable of generating a gas by reaction with a proton donor compound. The gas formed is carbon dioxide, oxygen or chlorine dioxide or any other type of biocompatible gas.

According to one advantageous embodiment the alkaline agent is selected from the group comprising potassium, lithium, sodium, calcium or ammonium carbonate or L-lysine carbonate, arginine carbonate, sodium glycine carbonate, the sodium carbonates of amino acids, anhydrous sodium perborate, effervescent perborate, sodium perborate monohydrate, sodium percarbonate, sodium dichloroisocyanurate, sodium hypochlorite, calcium hypochlorite, and mixtures thereof.

In the context of the present invention the term “carbonate” refers equally to carbonates, sesquicarbonates, and hydrogencarbonates.

The respective amounts of acidic agent and alkaline agent are adjusted so that the reaction between the alkaline agent and the protons released by the acid allows a sufficient quantity of gas to be generated to obtain a satisfactory effervescence.

According to one advantageous embodiment, and especially when the active principle has an unpleasant taste, an excess of alkaline agent or acidic agent is employed so as to maintain around the active principle a specific pH environment, an environment in which the taste of the active principle is attenuated.

According to one advantageous embodiment the acidic agent and the alkaline agent are employed in the effervescent granules in powder or granule form.

This effervescent mixture may be supplemented, according to one advantageous embodiment, by an internal dehydrating agent, whose function is to stabilize the product, particularly with regard to the amount of water present in the tablet, in order to avoid a triggering of the effervescence reaction in the course of storage of the product. The internal dehydrating agents used conventionally are monosodium carbonate, anhydrous trimagnesium dicitrate, or any other, pharmaceutically acceptable salt which is a water scavenger.

Said dehydrating agent may be introduced directly into the effervescent granules, or else it may be added in external phase to the other excipients of the tablet by powder mixing, or else it may be added partitioned between the effervescent granule and the mixture of excipients of the tablet.

The heat-extrudable binder is a binder which is sufficiently rigid at room temperature and pressure but which can be deformed or which is able to form a semiliquid material under elevated pressure or temperature.

According to one specific embodiment this binder has a melting or softening temperature of less than 150° C.

It is nevertheless possible to use binders which have a melting or softening temperature greater than 150° C. provided they are used in the presence of a plasticizer. The plasticizers which can be used in the present invention may be selected from the group comprising in particular low molecular weight polymers, oligomers, copolymers, oils, small organic molecules, low molecular weight polyols having aliphatic hydroxyl groups, ester-type plasticizers, glycol ethers, poly(propylene glycol), multiblock polymers, monoblock polymers, low molecular weight poly(ethylene glycol), citrate ester-type plasticizers, triacetin, propylene glycol, and glycerol.

These plasticizers may also be ethylene glycol, 1,2-butylene glycol, 2,3-butylene glycol, styrene glycol, diethylene glycol, thriethylene glycol, tetraethylene glycol, and other compounds of poly(ethylene glycol) type, monopropylene glycol monoisopropyl ether, propylene glycol monoethyl ether, sorbitol lactate, butyl lactate, ethyl glycolate, dibutyl sebacate, acetyl tributyl citrate, triethyl citrate, acetyl triethyl citrate, tributyl citrate, and allyl glycolate, and mixtures thereof.

It is also possible to introduce, when required, a pharmaceutical compatible antioxidant selected from the groups of the antioxygens, reducing agents or antioxygen synergistic agents. In particular such compounds include, for the first group, tocopherol derivatives, in the case of the reducing agents, ascorbic acid, and within the third group citric acid, tartaric acid, and lecithin.

The extrudable binder may be selected from the group comprising in particular tragacanth gum, gelatin, starch, cellulose derivatives such as sodium carboxymethylcellulose, methylcellulose, povidone derivatives, methacrylic acid derivatives, alginic acids and their salts, polyethylene glycols, guar gum, polysaccharides, sucrostearate, poloxamers (Pluronic F68, Pluronic F127), collagen, albumin, gelatin, and mixtures thereof.

Other binders are selected from the group comprising in particular propylene glycol, polyoxyethylene-poly-propylene copolymer, polyethylene ester, polyethylene sorbitan ester, polyethylene oxide, and the like.

According to one particularly advantageous embodiment the binder is a polyethylene glycol, preferably having a molecular mass of from 1000 to 8000 Da.

The binder may be used in any form whatsoever, particularly in the form of a powder, granules, chips, or in the melted state.

According to one advantageous embodiment the amount of binder used in the effervescent granules is less than 60%, preferably between 3 and 8% by weight relative to the weight of the effervescent granules.

Where the active principle is incorporated into the effervescent granules, and provided that the active principle is in acid form, the active principle may itself play the part of a proton donor for generating effervescence in the presence of the alkaline agent.

The active principle is in the form of microparticles, microgranules or any particle form constituted by a neutral core onto which the crystals of active principle are deposited.

Where the active principle is coated, the coating polymer is selected advantageously from the group comprising cellulosic polymers, acrylic polymers, vinyl polymers, waxes, and derivatives and mixtures thereof.

Among cellulosic polymers it will be advantageous to select ethycellulose, hydroxypropylcellulose (HPC), and hydroxypropylmethylcellulose (HPMC), alone or in a mixture, or cellulose acetophtalate.

Among acrylic polymers it will be advantageous to select amonio-methacrylate copolymer (Eudragit® RL and RS), polyacrylate (Eudragit® NE), and polymetacrylate (known in particular under the name Eudragit® E), Eudragit® being a trade mark registered by Röhm & Haas.

The coating polymer is present in proportions which can range up to 50% by weight, preferably from 10 to 35% by weight relative to the weight of the coated particles of active principle.

According to another advantageous embodiment the taste masking coating is an enteric coating.

The tablets in accordance with the invention comprise a mixture of excipients comprising

a disintegrant,

a diluent soluble agent having binding properties,

a lubricant, and

optionally an internal dehydrating agent, a swelling agent, a permeabilizing agent, sweeteners, flavorings and colorants.

According to one advantageous embodiment the excipient/coated or uncoated active principle ratio is from 0.4 to 6, preferably from 1 to 4.

The disintegrant is selected from the group comprising in particular crosslinked sodium carboxy-methylcellulose, referred to in the art as croscarmellose, crospovidone, starches and derivatives, such as pregelatinized modified starches and carboxymethylstarch, hydroxypropylcellulose derivatives with low degrees of substitution, and mixtures thereof.

The diluent soluble agent having binding properties is preferably constituted by a polyol having less than 13 carbon atoms which is present either in the form of a directly tabletable product whose average particle diameter is from 100 to 500 μm, or in the form of a powder whose average particle diameter is less than 100 μm, this polyol being selected preferably from the group comprising mannitol, lactitol, erythritol and derivatives, xylitol, sorbitol, and maltitol, with the proviso that sorbitol cannot be used alone and that, where the diluent soluble agent having binding properties is alone, it is used in the form of the directly tabletable product, whereas, where there are at least two diluent soluble agents having binding properties, one is present in the directly tabletable form and the other in the powder form, it being possible for the polyol to be the same, the proportions of directly tabletable polyol and of polyol powder being from 99/1 to 20/80, preferably from 80/20 to 20/80.

The respective proportions of disintegrant and soluble agent employed for the constitution of the excipient are, relative to the mass of the tablet, from 1 to 15%, preferably from 2 to 7% by weight for the former and from 30 to 85%, preferably from 40 to 70% by weight, for the latter.

The lubricant is selected from the group comprising in particular magnesium stearate, stearic acid, sodium stearylfumarate, micronized polyoxyethyleneglycol (micronized Macrogol 6000), leucine, talc, sodium benzoate, and mixtures thereof.

The amount of lubricant is from 0.2 to 20 parts per thousand (weight of lubricant/total weight of the tablet), preferably from 5 to 10 parts per thousand.

The lubricant may be distributed within the excipient; according to one advantageous embodiment, the totality of the lubricant may be distributed on the surface of the tablet (patent application WO 00/51568).

The lubricant may optionally be present in the effervescent granules.

As a permeabilizing agent it is advantageous to use a compound selected from the group comprising in particular silicas having a great affinity for aqueous solvents, such as the precipitated silica better known under the brand name Syloïd®, maltodextrins, β-cyclodextrins, and mixtures thereof.

The permeabilizing agent allows the creation of a hydrophilic network which promotes the penetration of saliva and thereby contributes to better disaggregation of the tablet.

The proportion of permeabilizing agent relative to the mass of the tablet is from 0% to 10% by weight.

The sweetener may be selected from the group comprising in particular aspartame, potassium acesulfam, sodium saccharinate, neohesperidine dihydrochalcone, sucralose, monoammonium glycyrrhizinate, and mixtures thereof.

The flavorings and colorants are those used commonly in pharmacy for the preparation of tablets.

Advantageously, and especially when the active principle has an acceptable taste or a taste which is linked to the pH environment, the tablet comprises:

2% to 85%, preferably from 3% to 40%, of effervescent granules comprising an uncoated active principle,

1% to 15%, preferably from 2% to 7%, of disintegrant,

0% to 30%, preferably from 2% to 15%, of dehydrating agent,

up to 1.5%, preferably from 0.25% to 1%, of lubricant,

0 to 10%, preferably from 0.5% to 5%, of permeabilizing agent,

0 to 3.5%, preferably from 0.5% to 3%, of colorant,

0 to 3%, preferably from 0.5% to 2.5%, of flavoring,

diluent soluble agent qsp 100%,

the percentages being calculated by weight relative to the total weight of the tablet.

When the active principle has a very unpleasant taste it is necessary to coat it with a taste masking coating. One tablet which meets this requirements comprises:

2% to 90%, preferably from 3% to 40%, of effervescent granules,

15% to 70%, preferably from 20% to 50%, of coated active principle,

1% to 15%, preferably from 2% to 7%, of disintegrant,

0% to 30%, preferably from 2% to 15%, of dehydrating agent,

up to 1.5%, preferably from 0.25% to 1%, of lubricant,

0 to 10%, preferably from 0.5% to 5%, of permeabilizing agent,

0 to 3.5%, preferably from 0.5% to 3%, of colorant,

0 to 3%, preferably from 0.5% to 2.5%, of flavoring,

diluent soluble agent qsp 100%,

the percentages being calculated by weight relative to the total weight of the tablet.

According to a second embodiment particularly appropriate to active principles having a very unpleasant taste, the tablet comprises:

15% to 70%, preferably from 20% to 50%, of effervescent granules comprising a coated active principle,

1% to 15%, preferably from 2% to 7%, of disintegrant,

0% to 30%, preferably from 2% to 15%, of dehydrating agent,

up to 1.5%, preferably from 0.25% to 1%, of lubricant,

0 to 10%, preferably from 0.5% to 5%, of permeabilizing agent,

0 to 3.5%, preferably from 0.5% to 3%, of colorant,

0 to 3%, preferably from 0.5% to 2.5%, of flavoring,

diluent soluble agent qsp 100%,

the percentages being calculated by weight relative to the total weight of the tablet.

The tablets according to the invention make it possible to obtain disintegration times within the mouth, under the action of saliva and in the absence of any chewing action, of less than 60 seconds and preferably less than 40 seconds. This disintegration time corresponds to the period which elapses between, on the one hand, the moment when the tablet is placed in the mouth in contact with saliva and, on the other hand, the moment when the suspension resulting from the disaggregation, without mastication, of the tablet in contact with the saliva is swallowed.

These tablets have a hardness and an abrasion which are sufficient to withstand the various operations required for their packaging and their storage.

They have satisfactory organoleptic qualities.

The invention will be even more readily understood with the aid of the examples which follow, and which are not limitative but relate to advantageous embodiments of the invention.

### EXAMPLE 1

### Effervescent Granules Containing no Active Principle, Prepared According to a First Procedure

10 types of effervescent granules, identified respectively by the letters A to J, are prepared, the compositions of which are given below.

These effervescent granules are prepared under a humidity-controlled atmosphere, with optional drying, prior to use of the components which are too moist. The various constituents identified below may be mixed beforehand in an external mixer or directly in the hot extruder.

Within the hot extruder the mixture is brought to a temperature of not more than approximately 120° C. at a rate, and for a time, which are sufficient to melt or soften the binder.

The extrudate is recovered and is chopped or ground.

The effervescent granules thus prepared are then screened with a screen having a mesh size of 710 microns, and then stored in a low-humidity atmosphere.

Granules A

| Constituents | Amounts (% by weight) |
|---|---|
| NaHCO₃ | 52 |
| Citric acid | 14 |
| Tartaric acid | 28 |
| PEG 1000 | 6 |

Granules B

| Constituents | Amounts (% by weight) |
|---|---|
| NaHCO₃ | 55 |
| Citric acid | 13.5 |
| Tartaric acid | 24 |
| PEG 4000 | 7.5 |

Granules C

| Constituents | Amounts (% by weight) |
|---|---|
| Sodium glycine carbonate | 58 |
| Citric acid | 15 |
| Tartaric acid | 21 |
| Pluronic F68 | 6 |

Granules D

| Constituents | Amounts (% by weight) |
|---|---|
| NaHCO₃ | 54 |
| Citric acid | 16 |
| Tartaric acid | 24 |
| PEG 20000 | 3 |
| PEG 400 | 3 |

Granules E

| Constituents | Amounts (% by weight) |
|---|---|
| NaHCO₃ | 50 |
| Citric acid | 14 |
| Tartaric acid | 28 |
| PEG 8000 | 8 |

Granules F

| Constituents | Amounts (% by weight) |
|---|---|
| KHCO₃ | 62 |
| Fumaric acid | 5 |
| Citric acid | 8 |
| Tartaric acid | 18 |
| PEG 6000 | 7 |

Granules G

| Constituents | Amounts (% by weight) |
|---|---|
| NaHCO₃ | 55 |
| NaH₂PO₄ | 37.5 |
| Pluronic F127 | 7.5 |

Granules H

| Constituents | Amounts (% by weight) |
|---|---|
| NaHCO₃ | 54 |
| Fumaric acid | 3 |
| Maleic acid | 5 |
| Citric acid | 13 |
| Tartaric acid | 18 |
| PEG 3350 | 6 |
| Pluronic F68 | 4 |

Granules I

| Constituents | Amounts (% by weight) |
|---|---|
| NaHCO₃ | 56 |
| Citric acid | 37 |
| Cetyl alcohol | 4 |
| Stearyl alcohol | 5 |

Granules J

| Constituents | Amounts (% by weight) |
|---|---|
| NaHCO₃ | 51 |
| Citric acid | 34 |
| Xylitol | 15 |

### EXAMPLE 2

### Preparation of Tablets Comprising Effervescent Granules and a Coated Active Principle

Coated granules of active principle are prepared whose percentage composition is as follows:

| | |
|---|---|
| Celecoxib | 85% |
| Precipitated silica | 1% |
| Ethylcellulose N7 | 12% |
| Hydroxypropylmethylcellulose | 2% |

These coated granules of active principle are employed in tablets in accordance with the invention having a celecoxib content of 100 mg or 200 mg and the following percentage composition:

| | Percentage |
|---|---|
| Ingredients | composition |
| Coated celecoxib granules | 40% |
| Effervescent granules A | 10% |
| Pulverulent mannitol | 15% |
| Granulated mannitol | 15% |
| Crospovidone | 8% |
| Precipitated silica | 3% |
| Aspartame | 1.7% |
| Potassium acesulfam | 0.9% |
| Mint flavorings | 0.35% |
| Magnesium stearate | 0.05% |

The various ingredients above are mixed and the resulting mixture is tableted using a rotary tableting machine.

The diameter of the tablets is 10 mm for the 100 mg dose and 13 mm for the 200 mg dose.

The tablets obtained are 294 mg tablets for the 100 mg dose and 588 mg tablets for the 200 mg dose.

They have a hardness comprised between 15 and 50 Newton.

The disintegration time in the mouth is less than 60 seconds. This disintegration time corresponds to the period which elapses between, on the one hand, the moment at which the tablet is placed in the mouth in contact with the saliva and, on the other hand, the moment at which the suspension resulting from the disaggregation, without chewing of the tablet in contact with the saliva is swallowed.

The mouthfeel of these tablets appeared satisfactory.

### EXAMPLE 3

### Preparation of Tablets Comprising Effervescent Granules and a Coated Active Principle

Coated granules of active principle are prepared whose percentage composition is as follows:

| | |
|---|---|
| Ondansetron | 75% |
| Precipitated silica | 1% |
| Eudragit E100 | 24% |

These coated granules of active principle are employed in tablets in accordance with the invention having a ondansetron content of 4 mg or 8 mg and the following percentage composition:

| | Percentage |
|---|---|
| Ingredients | composition |
| Coated ondansetron granules | 3% |
| Effervescent granule A | 28% |
| Pulverulent mannitol | 29% |
| Granulated mannitol | 29% |
| Crospovidone | 6% |
| Precipitated silica | 1% |
| Aspartame | 3.6% |
| Mint flavorings | 0.35% |
| Magnesium stearate | 0.05% |

The various ingredients above are mixed and the resulting mixture is tableted using a rotary tableting machine.

The diameter of the tablets is 8 mm for the 4 mg dose 15 and 10 mm for the 8 mg dose.

The tablets obtained are 183 mg tablets for the 4 mg dose and 366 mg tablets for the 8 mg dose.

They have a hardness comprised between 15 and 30 Newton.

The disintegration time in the mouth is less than 60 seconds. This disintegration time is measured as in example 2.

The mouthfeel of these tablets appeared satisfactory.

### EXAMPLE 4

### Preparation of Tablets Comprising Effervescent Granules Themselves Comprising an Uncoated Active Principle

Effervescent granules are prepared which have the following percentage composition:

| | |
|---|---|
| Loratadine | 7% |
| Sodium bicarbonate | 54% |
| Citric acid, anhydrous | 27% |
| Xylitol | 6% |
| Poloxamer | 6% |

These effervescent granules are prepared as in example 1 except that the active principle is incorporated into the pulverulent mixture.

The effervescent granules comprising an active principle are employed in tablets in accordance with the invention which have a loratadine content of 10 mg and the following percentage composition:

| | |
|---|---|
| Effervescent granules | 45% |
| containing loratadine | |
| Mannitol granules | 22% |
| Mannitol powder | 22% |
| Croscarmellose | 5% |
| Precipitated silica | 2% |
| Aspartame | 3% |
| Orange flavoring | 0.9% |
| Magnesium stearate | 0.1% |

The mixture thus obtained is tableted using a rotary tableting machine.

The diameter of the tablets is 9 mm.

The tablets obtained are 318 mg tablets having a hardness comprised between 20 and 50 Newton.

The disintegration time in the mouth is less than 60 seconds. This time is measured as in example 2.

The mouthfeel of these tablets appeared satisfactory.

### EXAMPLE 5

### Preparation of Tablets Comprising Effervescent Granules Themselves Comprising an Uncoated Active Principle

Tablets in accordance with the invention are prepared using as effervescent granules the granules A of example 1 and as active principle sildenafil

Three types of tablets are prepared, having a sildenafil dose of 25 mg, 50 mg and 100 mg, and each having the following percentage formula:

| | Percentage |
|---|---|
| Ingredients | composition |
| Sildenafil | 14.7% |
| Effervescent granules A | 23.8% |
| Mannitol powder | 24% |
| Mannitol granules | 24% |
| Crospovidone | 7% |
| Monosodium carbonate | 2.4% |
| Precipitated silica | 1.8% |
| Aspartame | 1.3% |
| Potassium acesulfam | 0.7% |
| Mint flavorings | 0.23% |
| Magnesium stearate | 0.07% |

The mixture thus obtained is tableted using a rotary tableting machine.

The diameter of the tablets is, respectively, 7 mm for a 25 mg dose, 10 mm for a 50 mg dose, and for a 100 mg dose.

The tablets obtained are 170 mg tablets for a 25 mg dose, 340 mg tablets for a 50 mg dose, and 680 mg tablets for a 100 mg dose.

They have a hardness comprised between 15 and 50 Newton.

The disintegration time in the mouth is less than 60 seconds. This time is measured as in example 2.

The mouthfeel of these tablets appeared satisfactory.

### EXAMPLE 6

### Preparation of Tablets Comprising Effervescent Granules Themselves Comprising Coated Methylprednisolone. (Dose: 5 mg)

Effervescent granules are prepared which contain methylprednisolone as active principle.

These granules are prepared as in example 1 except that the methylprednisolone is added to the pulverulent mixture. This gives effervescent granules having the following percentage composition:

| | |
|---|---|
| Coated methylprednisolone granules | 9.825% |
| Sodium bicarbonate | 45% |
| Citric acid, anhydrous | 35% |
| Microcrystalline cellulose | 8.175% |
| Sucrose stearate | 2% |

The coated methylprednisolone granules employed in the effervescent granules have the following percentage composition:

| | |
|---|---|
| Methylprednisolone | 80% |
| Precipitated silica | 4% |
| Eudragit NE30D | 16% |

The effervescent granules containing the coated methylprednisolone granules are employed in tablets in accordance with the invention having a methylprednisolone content of 5 mg and the following percentage composition:

| | Percentage |
|---|---|
| Ingredients | composition |
| Effervescent coated methylprednisolone | 50% |
| granules | |
| Mannitol granules | 20% |
| Mannitol powder | 20% |
| Croscarmellose | 4% |
| Precipitated silica | 2% |
| Aspartame | 3% |
| Orange flavoring | 0.9% |
| Magnesium stearate | 0.1% |

The various ingredients above are mixed and the mixture thus obtained is tableted using a rotary tableting machine.

The diameter of the tablets is 8 mm.

The tablets obtained are 200 mg tablets with a hardness comprised between 15 and 40 Newton.

The disintegration time in the mouth is less than 60 seconds. This time is measured as in example 2.

The mouthfeel of these tablets appeared satisfactory.

### EXAMPLE 7

### Preparation of Tablets Comprising Effervescent Granules Themselves Comprising Coated Methylprednisolone. (Dose: 20 mg)

Effervescent granules are prepared which contain methylprednisolone as active principle.

These granules are prepared as in example 1 except that 20 the methylprednisolone is added to the pulverulent mixture. This gives effervescent granules having the following percentage composition:

| | Percentage |
|---|---|
| Ingredients | composition |
| Coated methylprednisolone granules | 29.475% |
| Sodium bicarbonate | 22.4% |
| Citric acid, anhydrous | 38.125% |
| Microcrystalline cellulose | 8% |
| Sucrose stearate | 2% |

The coated methylprednisolone granules employed in the effervescent granules have the following percentage composition:

| | |
|---|---|
| Methylprednisolone | 80% |
| Precipitated silica | 4% |
| Eudragit NE30D | 16% |

The effervescent granules containing the coated methylprednisolone granules are employed in tablets in accordance with the invention having a methylprednisolone content of 20 mg and the following percentage composition:

| | |
|---|---|
| Effervescent coated methylprednisolone granules | 41.67% |
| Mannitol granules | 24.165% |
| Mannitol powder | 24.165% |
| Croscarmellose | 4% |
| Precipitated silica | 2% |
| Aspartame | 3% |
| Orange flavoring | 0.9% |
| Magnesium stearate | 0.1% |

The various ingredients above are mixed and the mixture thus obtained is tableted using a rotary tableting machine.

The diameter of the tablets is 10 mm.

The tablets obtained are 300 mg tablets with a hardness comprising between 15 and 40 newtons.

The disintegration time in the mouth is less than 60 seconds. This time is measured as in example 2.

The mouthfeel of these tablets appeared satisfactory.

1-7. (cancelled).

8. An orodispersible effervescent tablet comprising:
at least one active principle,
a mixture of excipients comprising at least one disintegrant, a diluent soluble agent, a lubricant and optionally an internal dehydrating agent, a swelling agent, a permeabilizing agent, sweeteners, flavorings and colorants,
and effervescent granules based on a mixture constituted essentially by an acidic agent, a heat-extrudable binder and an alkaline agent and optionally a dehydrating agent and a lubricant,
said granules being prepared by hot extrusion in the absence of water and solvents,
and said tablet undergoing disaggregation in the buccal cavity in contact with saliva in less than 60 seconds and preferably in less than 40 seconds.

9. An orodispersible effervescent tablet comprising:
a mixture of excipients comprising at least one disintegrant, a diluent soluble agent, a lubricant and optionally an internal dehydrating agent, a swelling agent, a permeabilizing agent, sweeteners, flavorings and colorants,
and effervescent granules based on at least one active principle and a mixture constituted by an acidic agent, a heat-extrudable binder and an alkaline agent and optionally a dehydrating agent and a lubricant,
said granules being prepared by heat extrusion in the absence of water and solvents,
and said tablet undergoing disaggregation in the buccal cavity in contact with saliva in less than 60 seconds and preferably in less than 40 seconds.

10. A tablet according to any one of claim 8 or 9, wherein the active principle is coated with a coating which gives it properties of taste masking, stabilization, gastric fluid resistance or modified release.

11. A tablet according to any one of claims 8 or 9, wherein the excipient mixture/active principle ratio is from 0.4 to 6, preferably from 1 to 4.

12. A tablet according to any one of claims 8 or 9, wherein said tablet comprises:
2% to 90%, preferably from 3% to 40%, of effervescent granules,
15% to 70%, preferably from 20% to 50%, of coated active principle,
1% to 15%, preferably from 2% to 7%, of disintegrant,
0% to 30%, preferably from 2% to 15%, of dehydrating agent,
up to 1.5%, preferably from 0.25% to 1%, of lubricant,
0% to 10%, preferably from 0.5% to 5%, of permeabilizing agent,
0% to 3.5%, preferably from 0.5% to 3%, of colorant,
0% to 3%, preferably from 0.5% to 2.5%, of flavoring,
diluent soluble agent qsp 100%,
the percentages being calculated by weight relative to the total weight of the tablet.

13. A tablet according to claim 9, wherein said tablet comprises:
2% to 85%, preferably from 3% to 40%, of effervescent granules comprising an uncoated active principle,
1% to 15%, preferably from 2% to 7%, of disintegrant,
0% to 30%, preferably from 2% to 15%, of dehydrating agent,
up to 1.5%, preferably from 0.25% to 1%, of lubricant,
0% to 10%, preferably from 0.5% to 5%, of permeabilizing agent,
0% to 3.5%, preferably from 0.5% to 3%, of colorant,
0% to 3%, preferably from 0.5% to 2.5%, of flavoring,
diluent soluble agent qsp 100%,
the percentages being calculated by weight relative to the total weight of the tablet.

14. A tablet according to claim 9, wherein said tablet comprises:
15% to 70%, preferably from 20% to 50%, of effervescent granules comprising a coated active principle,
1% to 15%, preferably from 2% to 7%, of disintegrant,
0% to 30%, preferably from 2% to 15%, of dehydrating agent,
up to 1.5%, preferably from 0.25% to 1%, of lubricant,
0 to 10%, preferably from 0.5% to 5%, of permeabilizing agent,
0 to 3.5%, preferably from 0.5% to 3%, of colorant,
0 to 3%, preferably from 0.5% to 2.5%, of flavoring,
diluent soluble agent qsp 100%,
the percentages being calculated by weight relative to the total weight of the tablet.
